(19) United States
(12) Patent Application Publication
(10) Pub. No.: US 2025/0241738 A1
(43) Pub. Date: Jul. 31, 2025

(54) Device, System, and Method for Prolapse Alleviation and/or Incontinence

(71) Applicant: CoreCreation UG, Berlin (DE)

(72) Inventors: Jair Kirah, Berlin (DE); Linda Wonneberger, Berlin (DE); Hugo Miguel Da Cunha Alves Ferreira Da Silva, Berlin (DE); Kaven Baessler, Berlin (DE)

(21) Appl. No.: 18/720,761

(22) PCT Filed: Dec. 20, 2022
(86) PCT No.: PCT/EP2022/086940

§ 371 Date: Jun. 17, 2024

(30) Foreign Application Priority Data
Dec. 22, 2021 (EP) 21217045.0

(51) Int. Cl. A61F 2/00

(52) U.S. Cl. CPC A61F 2/0013; A61F 2230/0008; A61F 2230/0065; A61F 2250/0036

(57) ABSTRACT

The present invention relates to a system with a device for alleviating prolapses and/or incontinence, comprising a support component or anterior end that is configured to provide at least partial urethral support. This is intended to mean to exert pressure onto the urethra of a female in use, compress the urethra or even close it. Further, there the device or support component is configured to be inserted into a vagina and to be expandable.

### FIELD

The invention relates to a device, system and a method for treating or preventing pelvic organ prolapse and/or urinary incontinence.

### INTRODUCTION

Pelvic organ prolapse (POP) is characterized by descent of pelvic organs from their normal positions. In women, the condition usually occurs when the pelvic floor connective tissue and muscles weaken and their support collapses during pregnancy or after childbirth. Apart from pregnancy and childbirth hormonal changes during menopause, obesity, family history of pelvic floor disorders and occupational heavy lifting are risk factors. In men, rectal prolapse can occur and incontinence may develop after the prostate gland is removed.

Defects in the fascial membranes and other connective structures like ligaments as well as structural and functional pelvic floor muscle damage that result in cystocele, rectocele, enterocele and uterine prolapse (or so-called vault prolapse when the uterus had been removed) POP may include one or more of the following conditions:
a) Cystocele, the most common form of pelvic organ prolapses, wherein the bladder drops into the vagina and may be associated with urination problems like incontinence and sometimes bladder emptying problems and bladder infections;
b) Rectocele, wherein the rectum herniates into the vagina and may result in difficulty and/or pain with defecation as well as faecal incontinence;
c) Enterocele, wherein the small intestine prolapses into the vagina;
d) Uterine prolapse wherein the uterus drops downward into or outside of the vagina; and
e) Vaginal vault prolapse, wherein the top portion of the vagina, the apex, loses its natural shape and drops down into the lower vaginal canal, and may only occur in women who had a hysterectomy.

A treatment can involve dietary and lifestyle changes, physical therapy, or surgery. Since surgery is associated with side effects including pain, dyspareunia (pain during sexual intercourse), infection, organ perforation and recurrence, conservative measures such as achieving normal weight and fitness, pelvic floor muscle exercises and insertion of support devices like pessaries are preferred. With a pessary, a rubber or silicone device is inserted into the vagina and may be retained for up to several months. Pessaries are a good choice of treatment for women who wish to maintain fertility, are poor surgical candidates, or who do not want to undergo surgery. Several pessaries are known, for example: ring pessary, shelf pessary, cube pessary, Gelhorn pessary, Hodge pessary etc.

WO9601084A1 discloses a pessary device, comprising a first inflatable chamber, adapted to fit in the vaginal cavity of a woman when inflated, wherein the chamber compresses the urethra of the woman when inflated; an anchor member, being connected to the inflatable chamber on the anterior side of the chamber when the pessary is in its normal operating position; and an inflation tube having a posterior end fluidly connected to the inflatable chamber and a second end connected to an inflation valve located external to the vagina when the pessary device is in its normal operating position that permits inflation and deflation of the inflatable chamber without disturbing the fit of the anchor member.

GB2352181A discloses an inflatable pessary adapted to be placed in a female vagina comprising elongate support means, inflatable restriction means being connected to the support means, and an inflation valve located remote from said inflatable restriction means, wherein the restriction means is arranged such that when inflated, pressure is applied outwardly to the walls of the vagina. The pessary may also include a second inflatable restriction means where the restriction means may be independently deflated and inflated. The pessary may be used to provide artificial continence, to be used as a perineometer (vaginal manometer is an instrument for measuring the strength of voluntary contractions of the pelvic floor muscles) or to provide sexual stimulation.

EP2276419B1 discloses devices for supporting a prolapsed organ. An exemplary device comprises a ring-like body optionally having a naturally occurring substantially flat and substantially planar compact configuration, the ring-like body configured with a size suitable for insertion into a vagina and to be expanded by a support element such that in the expanded configuration an outer periphery of the ring-like body contacts a portion of the vagina and stretches at least a portion of a prolapsed vaginal wall, thereby substantially alleviating prolapse of at least one pelvic organ. The device optionally includes a support element comprising two moveably connected arms configured to support the ring in the expanded configuration.

### SUMMARY

It is the object of the present invention to ameliorate the shortcomings and provide another or modular and easy to use device, system and/or method for alleviating prolapses and/or incontinence.

The method described can be also performed without the presence of any user for test reasons or simulation reasons with a dummy or without any other component.

The present invention relates to a device for alleviating prolapses and/or incontinence, comprising a support component that is configured to provide at least partial urethral support. This is intended to mean to exert pressure onto the urethra of a female in use, compress the urethra or even close it. Further, there the device or support component is configured to be inserted into a vagina and to be expandable.

The device can comprise an anterior end and a posterior end with two sides connecting the ends. This part of the device is also called body. Any one of the ends and/or the sides can have the form of ridges or hollow tube-like sections. This will be further specified below. The device can also be called a pessary (with a particular shape and/or functionalities).

The anterior end can be thicker and/or can have a larger cross-section than the posterior end and wherein the anterior end can be configured to support and/or compress a urethra from a vaginal side and the posterior end and the sides can be configured to support the device in the other parts of a vagina and/or cervix.

The device can comprise at least one expandable volume or cavity. The device can comprise at least one inflatable chamber or a chamber that can be filled with another medium, fluid or mixture of fluids. It may be noted that the at least one chamber can be further divided into different sections.

The device can comprise at least one inflatable chamber(s) within an anterior end and two sides of the device. The inflatable chamber(s) can be provided in each side and a posterior end of the device.

The device can comprise three inflatable chambers, one in each one of two sides and one in an anterior end.

The device can comprise silicone material, such as biocompatible silicone material. The support component can comprise a body. The body can comprise a generally torus shape, a generally toroidal shape, an oval ring shape, an elliptical ring shape, an ovoid shape, a V-shape, a heart shape and/or a trapezoid shape. Further, the body can comprise more than one compartment.

The body can comprise a posterior end, an anterior end as well as two sides or side ridges connecting these ends.

The body can comprise an external length (A), wherein the external length comprises the diameter of the body along a semi-major axis. The body can comprise an external width (D), wherein the external width comprises the diameter of the body along the semi-minor axis. The body can comprise an internal length (B), wherein the internal length comprises a diameter of an annulus of the body along the semi-major axis. The body can comprise an internal width (C), wherein the internal width comprises a diameter of an annulus of the body along the semi-minor axis.

The support component can be configured with at least one air chamber. The support component can be configured with three air chambers. The air chamber can be configured to be placed inside the body, such that when the air chamber is filled with air it causes an enlargement of the support component. The state of the support component can comprise at least one of at least an inflated state, at least a deflated state, at least a semi-inflated state, and/or at least a semi-deflated state.

The inflated state comprises all the air chambers filled with air. The deflated state can comprise all the air chambers emptied with air. The semi-inflated state can comprise the at least one air chamber filled with air. The semi-deflated state can comprise the at least one air chamber emptied with air.

The external length can be configured to be in the range of 40 mm to 100 mm, such that 50 mm to 80 mm, preferably, 55 mm to 75 mm in the deflated state. The external width can be configured to be in the range of 20 mm to 80 mm, such that 30 mm to 60 mm, preferably, 40 mm to 55 mm in the deflated state. The internal length can be configured to be in the range of 15 mm to 70 mm, such that 20 mm to 50 mm, preferably, 30 mm to 35 mm in the deflated state.

The internal width can be configured to be in the range of 40 mm to 100 mm, such that 50 mm to 80 mm, preferably, 55 mm to 75 mm in the deflated state.

In the inflated state the dimensions are enlarged and can be adapted to the individual anatomy of the female user.

The body can comprise an inflatable external length (E), wherein the inflatable external length comprises at least one portion of the external length along a semi-major axis.

The inflatable external length can be configured to be in the range of 10 mm to 40 mm, such that 15 mm to 30 mm, preferably, 15 mm to 20 mm in the deflated state.

The inflatable external length can be configured to be in the range of 15 mm to 50 mm, such that 20 mm to 30 mm, preferably, 25 mm in the inflated state.

The external width can be configured to be in the range of 30 mm to 90 mm, such that 40 mm to 70 mm, preferably, 50 mm to 65 mm in the inflated state.

A relation of a cross-sections of an anterior end (2c) to a cross-section of the posterior end (2a) can amount to at least 1.5, preferably at least 2 and more preferably at least 3.

The device can comprise a recess throughout the device and the projection of the area of the recess from a top view shown to the respective projection of the remaining part of the device amounts to a ratio of at least 0.1, preferably at least 0.2 and a maximum of 0.5, preferably 0.4 and more preferably 0.3.

The material and material parameters (such as thickness of material) can be configured to close the recess in expanded condition of the device when needed. The material can also have a higher rigidity.

An outer material with a texture that can be configured to support the holding of the device in a vagina.

The device can further comprise at least one sensor, such as at least one sensor for pressure, temperature and/or electromyographic (EMG).

An attachment component for a menstruation collecting component can also be arranged, such as a menstruation collection cup.

The support component can be configured integral with the connecting component. The connecting component can be configured to be an extension of the support component.

The connecting component can further comprise a releasing valve at the open end that can be configured to release fluid when being activated by a user. The releasing valve can be configured to be activated by a compression of a user. The releasing valve can further be activated by a manual intervention, i.e., by pushing a button or rotating a knob.

The connecting component can be configured to be closed by a cover and/or plug and/or filter in order to prevent any fluids entering the connecting component in an unwanted manner.

The connecting component can be configured to be folded onto or towards the device when closed and/or when the device is expanded. Further, a shape may be comprised that is like a Zigzag and/or spiral that can be expended by the user and then elastically come back to its compact position.

The connecting component can comprise an inflation tube. The connecting component can be further configured with at least one connecting valve.

The at least one connecting valve can be configured to be connected to a controlling component. Also, electrical connections may be comprised like electrical wiring and/or connectors.

The inflation tube can comprise silicone and/or latex and/or plastic material.

The at least one connecting valve can be configured to be covered by a covering that can further be configured to be removable. The covering can be configured to envelope at least a portion of a connecting valve opening.

The length of the connecting component can be configured to amount to 55 mm or can be configured to be in a range of 20 mm to 150 mm, such that 40 mm to 80 mm, such that 55 mm.

The connecting component can be configured to be foldable along the length of the inflation tube. Also, electrical connections may be comprised like electrical wiring and/or connectors. The thickness of the connecting component can amount to 3 mm. The thickness of the connecting component can be in a range of 2 mm to 10 mm.

The connecting component can be further configured with a control element and further configured to trigger an event such as release of air.

The connecting component can comprise at least one or a plurality of inflation tubes. The connecting component can comprise three inflation tubes. The at least one inflation tube can be connected with the at least one air chamber. Each of the three-inflation tubes can be connected with each of the three-air chamber.

Each of the inflation tube can be formed in the connecting valve. The controlling component can be configured with a plurality of valves. Each valve can be connected with the connecting valve, respectively.

The present invention is also directed to a system or assembly with multiple components. The system is also arranged for alleviating prolapses and/or incontinence. It can comprise a device according to the preceding and/or below description. The controlling component (3) can be configured to be coupled to the device for expanding or shrinking it.

The controlling component can comprise a pump for pumping fluid into the device and expanding it in a controlled manner. The pump can be an air pump, such as an electric air pump. The pump can also reduce fluid and shrink the one or more air chambers.

The controlling component can comprise at least one valve.

The controlling component can be further be configured to control liquid flow to the device with a pre-set maximum pressure.

The controlling component can be further configured with at least one user interface. The controlling component can comprise at least one sensor, such as pressure sensor for measuring the actual pressure delivered to the device or within the device.

The controlling component can comprise a battery.

The device and/or the controlling element can have a communication component communicating to a handheld device a status of the device and/or controlling element. This can communicate to a handheld device and receiving activation and/or de-activation commands from the handheld device.

The communication component can be configured to communicate wireless, such as by Bluetooth.

A method according to the present invention can be also used for testing and/or analysing the device and/or the system as described and can comprise an expansion of the device.

The step of measuring and/or analyzing the device can be done in expanded condition and/or in unexpanded condition.

The step of coupling the device and the controlling component and/or de-coupling it therefrom can be performed as well.

The step of folding or attaching the connecting component onto the device when the device is de-coupled from the controlling component can be taken as well.

The present invention is also directed to a method for a therapy of a female with the device and/or the system as described herein. An inserting of the device into a vagina of the female with an anterior end coming into neighbourhood of the female's urethra, expanding a device according to any of the preceding embodiments. A further step of decoupling the device with the connecting component from the remaining controlling component can be taken when the device is entered into a vagina. A further step of folding the connecting component onto the device can then be taken. Another further step of exercising a pelvic floor muscles of the female can be taken during presence of the device.

A removal of the device can result in a further step of unfolding the connecting component from the device and releasing air from the device and removing it from the female's vagina.

The present invention results in the following preferred advantages:

The device can support the weakening of the ligaments. Training of the pelvic muscles while using the support of the device reduces the weight from the ligaments and helps in their healing. With no weight on them, the ligaments can get shorter with time and hold the pelvic organs better.

Moreover, the shape as described can help entering the device with the assistance of the controlling component into a vagina in uninflated and rather flexible condition. The shape can further assist in holding the device in the vagina in an inclined and expanded condition. This is more endurable, and self-blocking by a positive locking effect and more comfortable for a user.

The recess in the device can allow body fluids to leave the body. However, the material and its properties can be chosen that the recess closes, if expanded and preferred.

By inflating the device, it can adapt itself to the individual anatomy of the user and the user can control this according to own needs easily.

The present invention is also defined by the following numbered embodiments.

Below is a list of device embodiments. Those will be indicated with a letter “D”. Whenever such embodiments are referred to, this will be done by referring to “D” embodiments.
D1. A device for alleviating prolapses and/or incontinence, comprising:
   a. a support component (2), configured to provide at least partial urethral support;
   b, wherein the support component (2) is configured to be inserted into a vagina and to be expandable.
D2. The device according to the preceding device embodiment wherein the device comprises a posterior end (2a) and a anterior end (2c) with two sides (2b) connecting the ends (2a, 2c).
D3. The device according to the preceding device embodiment wherein the anterior end (2c) is thicker and/or has a larger cross-section than the posterior end (2a) and wherein the anterior end (2c) is configured to support and/or compress a urethra from a vaginal side and the posterior end (2a) and the sides are configured to support the device in the other parts of a vagina.
D4. The device according to any of the preceding device embodiments wherein the device comprises at least one expandable volume.
D5. The device according to any of the preceding device embodiments wherein the device comprises at least one inflatable chamber.
D6. The device according to any of the preceding device embodiments wherein the device comprises at least one inflatable chamber(s) within a anterior end (2c) and two sides (2b) of the device.
D7. The device of the preceding device embodiment wherein the chamber(s) are provided in each side (2b) and an anterior end (2c) of the device.
D8. The device according to any of the preceding device embodiments wherein the device comprises three chambers, one in each one of two sides (2b) and one in an anterior end (2c).
D9. The device according to any of the preceding device embodiments wherein the device comprises more than three chambers, one in each one of two sides (2b) and one in an anterior end (2c).
D10. The device according to any of the preceding device embodiments wherein the chamber is adapted to be inflatable, one in each one of two sides (2b) and one in an anterior end (2c).
D11. The device according to any of the preceding device embodiments wherein the device comprises silicone material.
D12. The device according to any of the preceding device embodiments wherein the device comprises biocompatible silicone material.
D13. The device according to any of the preceding device embodiments wherein the support component comprises a body (2a-c).
D14. The device according to any of the preceding device embodiments wherein the body (2a-c) comprises a generally torus shape.
D15. The device according to any of the preceding device embodiments wherein the body (2a-c) comprises a generally toroidal shape.
D16. The device according to any of the preceding device embodiments wherein the body (2a-c) comprises an oval ring shape.
D17. The device according to any of the preceding device embodiments wherein the body (2a-c) comprises an elliptical ring shape.
D18. D7. The device according to any of the preceding device embodiments wherein the body (2a-c) comprises an ovoid shape.
D19. The device according to any of the preceding device embodiments wherein the body (2a-c) comprises a trapezoid shape.
D20. The device according to any of the preceding device embodiments wherein the body (2a-c) comprises a posterior end (2a), an anterior end (2c) as well as two sides or side ridges (2b) connecting these ends (2a,c).
D21. The device according to any of the preceding device embodiments wherein the body (2a-c) comprises a V shape.
D22. The device according to any of the preceding device embodiments wherein the body (2a-c) comprises a heart shape.
D23. The device according to any of the preceding device embodiments wherein the body comprises an external length (A), wherein the external length comprises the diameter of the body along a semi-major axis.
D24. The device according to any of the preceding device embodiments wherein the body comprises an external width (D), wherein the external width comprises the diameter of the body along the semi-minor axis.
D25. The device according to any of the preceding device embodiments wherein the body comprises an internal length (B), wherein the internal length comprises a diameter of an annulus of the body along the semi-major axis.
D26. The device according to the preceding device embodiment wherein the internal length comprises at least 15 mm, such that 20 mm, preferably 35 mm.
D27. The device according to any of the preceding device embodiments wherein the internal length comprises at most 70 mm.
D28. The device according to any of the preceding device embodiments wherein the body comprises an internal width (C), wherein the internal width comprises a diameter of an annulus of the body along the semi-minor axis.
D29. The device according to the preceding embodiment wherein the internal width comprises at least 10 mm, such that 20 mm, preferably 25 mm.
D30. The device according to any of the preceding embodiments wherein the internal width comprises at most 60 mm, such that 40 mm, preferably 25 mm.
D31. The device according to any of the preceding device embodiments wherein the support component is configured with at least one air chamber.
D32. The device according to any of the preceding device embodiments wherein the support component is configured with three air chambers.
D33. The device according to any of the preceding device embodiments wherein the air chamber is configured to be placed inside the body, such that when the air chamber is filled with air it causes an enlargement of the support component.
D34. The device according to any of the preceding device embodiments wherein the state of the support component comprises at least one of
   a. at least an inflated state, and
   b. at least a deflated state, and
   c. at least a semi-inflated state, and
   d. at least a semi-deflated state.
D35. The device according to any of the preceding device embodiments wherein the inflated state comprises all the air chambers filled with air.
D36. The device according to any one of the preceding device embodiments wherein the body comprises an external length (A), wherein the external length comprises the diameter of the body along a semi-major axis.
D37. The device according to any one of the preceding embodiments wherein the body comprises the external length (A) of up to 100 mm, preferably around to 75 mm, such that the external length comprises 110 mm to 65 mm.
D38. The device according to any of the preceding embodiments wherein the external length comprises a length of at least 40 mm.
D39. The device according to any one of the preceding embodiments wherein the body comprises an external width (D) wherein the external width comprises the diameter of the body along the semi-minor axis.
D40. The device according to any one of the preceding embodiments wherein the body comprises the external width (D) of up to 80 mm, preferably to around 70 mm.
D41. The device according to any of the preceding embodiments wherein the body comprises the external width of 110 mm in the inflated state.
D42. The device according to any of the preceding device embodiments wherein the external width (D) comprises of at least 20 mm.
D43. The device according to any of the preceding device embodiments wherein the deflated state comprises all the air chambers emptied with air, at least partially emptied with air.
D44. The device according to any of the preceding device embodiments wherein the semi-inflated state comprises the at least one air chamber filled with air.
D45. The device according to any of the preceding device embodiments wherein the semi-deflated state comprises the at least one air chamber emptied with air.
D46. The device according to any of the preceding device embodiments wherein the external length (A) is configured to be in the range of 40 mm to 100 mm, such that 50 mm to 80 mm, preferably, 55 mm to 75 mm in the deflated state.
D47. The device according to any of the preceding device embodiments wherein the external length (A) is configured to be in the range of 40 mm to 110 mm, such that 50 mm to 80 mm, preferably, 55 mm to 65 mm in the deflated state.
D48. The device according to any of the preceding device embodiments wherein the external width (D) is configured to be in the range of 20 mm to 80 mm, such that 30 mm to 60 mm, preferably, 40 mm to 55 mm in the deflated state.
D49. The device according to any of the preceding device embodiments wherein the external width (D) is configured to be in the range of 20 mm to 110 mm, such that 30 mm to 60 mm, preferably, 40 mm to 65 mm in the deflated state
D50. The device according to any of the preceding device embodiments wherein the internal length is configured to be in the range of 10 mm to 70 mm, such that 20 mm to 50 mm, preferably, 30 mm to 35 mm in the deflated state.
D51. The device according to any of the preceding device embodiments wherein the internal width is configured to be in the range of 40 mm to 100 mm, such that 50 mm to 80 mm, preferably, 55 mm to 75 mm in the deflated state.
D52. The device according to any of the preceding device embodiments wherein the body comprises an inflatable external length (E), wherein the inflatable external length comprises at least one portion of the external length along a semi-major axis.
D53. The device according to any of the preceding device embodiments wherein the inflatable external length is configured to be in the range of 10 mm to 40 mm, such that 15 mm to 30 mm, preferably, 15 mm to 20 mm in the deflated state.
D54. The device according to any of the preceding device embodiments wherein the inflatable external length is configured to be in the range of 15 mm to 50 mm, such that 20 mm to 30 mm, preferably, 25 mm in the inflated state.
D55. The device according to any of the preceding device embodiments wherein the external width is configured to be in the range of 30 mm to 90 mm, such that 40 mm to 70 mm, preferably, 50 mm to 65 mm in the inflated state.
D56. The device according to any of the preceding device embodiments wherein a relation of a cross-sections of a anterior end (2c) to a cross-section of the posterior end (2a) amounts to at least 1.5, preferably at least 2 and more preferably at least 3.
D57. The device according to any of the preceding device embodiments wherein the device comprises a recess throughout the device and the projection of the area of the recess from a top view shown to the respective projection of the remaining part of the device amounts to a ratio of at least 0.1, preferably at least 0.2 and a maximum of 0.5, preferably 0.4 and more preferably 0.3.
D58. The device according to any of the preceding device embodiments further comprising an outer material with a texture that is configured to support the holding of the device in a vagina.
D59. The device according to any of the preceding device embodiments further comprising at least one sensor.
D60. The device according to any of the preceding device embodiment further comprising at least one sensor for pressure, temperature and/or electromyographic (EMG).
D61. The device according to any of the preceding device embodiment further comprising at least one sensor for PH, motion, accelerometer, and/or gyroscope.
D62. The device according to any one of the preceding device embodiments configured for a training of biofeedback and learning about activated muscles inside a pelvic floor.
D63. The device according to any of the preceding device embodiment further comprising an attachment component for a menstruation collecting component.
D64. The device according to any of the preceding device embodiment further comprising an attachment component for a menstruation collecting cup.
D65. The device according to any of the preceding device embodiment further comprising a menstruation collecting component, such as a cup.
D66. The device according to any of the preceding device embodiments wherein the support component (2c) is configured integral with the connecting component (4).
D67. The device according to any of the preceding device embodiments wherein the material and/or material parameters are configured to close a recess in expanded condition of the device.
D68. The device according to the preceding device embodiment wherein the material and/or material parameters can comprise any of thickness and rigidity of material.
D69. The device according to any one of the preceding device embodiments wherein more than one material is comprised, wherein the materials have differing properties.
D70. The device according to any of the two preceding embodiments wherein the material in the vicinity of the recess comprises a second material different from a first material of the remaining device.
D71. The device according to any of the preceding device embodiments wherein the connecting component (4) is configured to be an extension of the support component (2c).
D72. The device according to any of the preceding device embodiments, the connecting component (4) further comprising a releasing valve at the open end that is configured to release fluid when being activated by a user.
D73. The device according to the preceding device embodiment wherein the releasing valve is configured be activated by a compression of a user.
D74. The device according to any of the preceding device embodiments wherein the connecting component (4) is configured to be closed by a cover and/or plug in order to prevent any fluids entering the connecting component in an unwanted manner.
D75. The device according to any one of the preceding device embodiments wherein the fluid is a contaminator.
D76. The device according to any of the preceding device embodiments wherein the connecting component (4) is configured to be folded onto or towards the device when closed and/or when the device is expanded.
D77. The device according to any of the preceding device embodiments wherein the connecting component (4) comprises an inflation tube.
D78. The device according to any of the preceding device embodiments wherein the connecting component is further configured with at least one connecting valve.
D79. The device according to any of the preceding device embodiments wherein the connecting component is configured as an electrically conducting thread.
D80. The device according to any of the preceding device embodiments wherein the at least one connecting valve is configured to be connected to a controlling component (3).
D81. The device according to any of the preceding device embodiments wherein the inflation tube comprises silicone.
D82. The device according to any of the preceding device embodiments wherein the inflation tube comprises latex.
D83. The device according to any of the preceding device embodiments wherein the inflation tube comprises plastic.
D84. The device according to any of the preceding device embodiments wherein the at least one connecting valve is configured to be covered by a covering.
D85. The device according to any of the preceding device embodiments wherein the covering is configured to be removable.
D86. The device according to any of the preceding device embodiments wherein the covering is configured to envelope at least a portion of a connecting valve opening.
D87. The device according to any of the preceding device embodiments wherein length of the connecting component is configured to amount to 55 mm.
D88. The device according to any of the preceding device embodiments wherein length of the connecting component is configured to be in a range of 30 mm to 100 mm, such that 40 mm to 80 mm, such that 55 mm.
D89. The device according to any of the preceding device embodiments wherein length of the connecting component is configured to be in a range of 20 mm to 150 mm, such that 55 mm.
D90. The device according to any of the preceding device embodiments wherein the connecting component is configured to be foldable along the length of the inflation tube.
D91. The device according to any of the preceding device embodiments wherein thickness of the connecting component amounts to 3 mm.
D92. The device according to any of the preceding device embodiments wherein the thickness of the connecting component is a range of 2 mm to 5 mm, such that 3 mm.
D93. The device according to any of the preceding device embodiments wherein the thickness of the connecting component is a range of 1 mm to 25 mm.
D94. The device according to any of the preceding device embodiments wherein the connecting component is further configured with a control element, further configured to trigger an event such as release of air.
D95. The device according to any of the preceding elements wherein the connecting component (4) comprises at least one plurality of inflation tubes.
D96. The device according to any of the preceding device embodiments wherein the connecting component (4) comprises three inflation tubes.
D97. The device according to any of the preceding device embodiments wherein the at least one inflation tube is connected with the at least one air chamber.
D98. The device according to any of the preceding device embodiments wherein each of the three-inflation tube is connected with each of the three-air chamber.
D99. The device according to any of the preceding device embodiments wherein each of the inflation tube is formed in the connecting valve.
D100. The device according to any of the preceding device embodiments wherein the controlling component is configured with a plurality of valves.
D101. The device according to any of the preceding device embodiments wherein each valve is connected with the connecting valve, respectively.

Below is a list of system embodiments. Those will be indicated with a letter “S”. Whenever such embodiments are referred to, this will be done by referring to “S” embodiments.
S1. A system for alleviating prolapses and/or incontinence, comprising:
   a device (2,4) according to any of the preceding device embodiments; and
   a controlling component (3) that can be coupled to the device for expanding it.
S2. The system according to any of the preceding system embodiments wherein the controlling component (3) comprises a pump for pumping fluid into the device and expanding it in a controlled manner.
S3. The system according to the preceding system embodiment wherein the pump is an air pump.
S4. The system according to the preceding system embodiment wherein the pump is an electric air pump.
S5. The system according to any of the preceding system embodiments wherein the controlling component comprises at least one valve.
S6. The system according to any of the preceding system embodiments wherein the controlling component (3) is further configured to control liquid flow to the device or back with a pre-set maximum pressure.
S7. The system according to any of the preceding system embodiments wherein the controlling component (3) is further configured with at least one user interface (3a).
S8. The system according to any of the preceding system embodiments wherein the controlling component comprises at least one sensor, such as pressure sensor for measuring the actual pressure delivered to the device or within the device.
S9. The system according to any one of the preceding system embodiments wherein the device is configured to support the pelvic floor muscles.
S10. The system according to any one of the preceding system embodiments wherein the device is configured to exercise the pelvic floor muscles.
S11. The system according to any of the preceding system embodiments wherein the controlling component further comprises at least one sensor for PH, motion, accelerometer, and/or gyroscope.
S12. The system according to any one of the preceding system embodiments wherein the controlling component is further configured for a training of biofeedback and learning about activated muscles inside a pelvic floor.
S13. The system according to any of the preceding system embodiments wherein the controlling component comprises a battery.
S14. The system according to any of the preceding system embodiments wherein the device (2) and/or the controlling element (3) has/have a communication component communicating to a handheld device a status of the device and/or controlling element.
S15. The system according to any of the preceding system embodiments wherein the device (2) and/or the controlling element (3) has/have a communication component communicating to a handheld device and receiving activation and/or de-activation commands from the handheld device.
S16. The system according to any of the two preceding system embodiments wherein the communication component is configured to communicate wireless, such as by Bluetooth.

Below is a list of method embodiments. Those will be indicated with a letter “M”. Whenever such embodiments are referred to, this will be done by referring to “M” embodiments.
M1. A method for testing and/or analysing the device according to any of the preceding device embodiments and/or the system according to any of the preceding system embodiments, comprising an expansion of the device (2,4).
M2. The method according to the preceding method embodiment further with the step of measuring and/or analysing the device (2,4) in expanded condition and/or in unexpanded condition.
M3. The method according to any of the preceding method embodiments further with the step of coupling the device (2,4) and the controlling component (3) and/or de-coupling it therefrom.
M4. The method according to any of the preceding method embodiments further with the step of folding or attaching the connecting component (4) onto the device (2) when the device is de-coupled from the controlling component (3).

Below is a list of therapeutical method embodiments. Those will be indicated with a letter “T”. Whenever such embodiments are referred to, this will be done by referring to “T” embodiments.
T1. A method for a therapy of a female with the device according to any of the preceding device embodiments and/or the system according to any of the preceding system embodiments.
T2. The therapeutical method according to the preceding therapeutical method comprising the steps of inserting a system according to any of the preceding embodiments into a vagina of the female with an anterior end coming into neighbourhood of the female's urethra, expanding a device according to any of the preceding embodiments.
T3. The treatment method according to the preceding treatment method with the further step of decoupling the device with the connecting component from the remaining controlling component.
T4 The treatment method according to the preceding treatment method with the further step of folding the connecting component onto the device.
T5. The treatment method according to the preceding treatment method with the further step of exercising pelvic floor muscles of the female.
T6 The treatment method according to any of the preceding treatment method with the further step of unfolding the connecting component from the device and releasing air from the device and removing it from the female's vagina.

Below, use embodiments will be discussed. These embodiments are abbreviated by the letter “U” followed by a number. Whenever reference is herein made to “use embodiments”, these embodiments are meant.
U1. Use of the system according to any of the preceding system embodiments for alleviating prolapses and/or incontinence.
U2. Use of the device according to any of the preceding device embodiments for alleviating prolapses and/or incontinence.

Below, computer related product embodiments will be discussed. These embodiments are abbreviated by the letter “C” followed by a number. Whenever reference is herein made to “computer related product embodiments”, these embodiments are meant,
C1. A computer related product with a program that is configured for carrying out the method according to any of the preceding method embodiments.

The present invention will now be described with reference to the accompanying drawings, which illustrate embodiments of the invention. These embodiments should only exemplify, but not limit, the present invention.

### BRIEF DESCRIPTION OF THE DRAWINGS

FIG. 1 exemplifies an embodiment of a device for alleviating prolapses and/or incontinence.

FIG. 2 shows a top view perspective onto the embodiment according to FIG. 1.

FIG. 3 shows a side view perspective onto the embodiment according to FIGS. 1 and 2.

FIG. 4 exemplifies a device according to the embodiment of any of the preceding figures in an inserted configuration with an activation of a hand by a user.

FIG. 5 shows the device according to FIG. 4 in a later stage when being decoupled to an activation component.

FIG. 6 depicts the device according to FIGS. 4 and/or 5 in a decoupled configuration during use.

FIG. 7 is an enlarged portion of FIG. 6.

### DESCRIPTION OF EMBODIMENTS

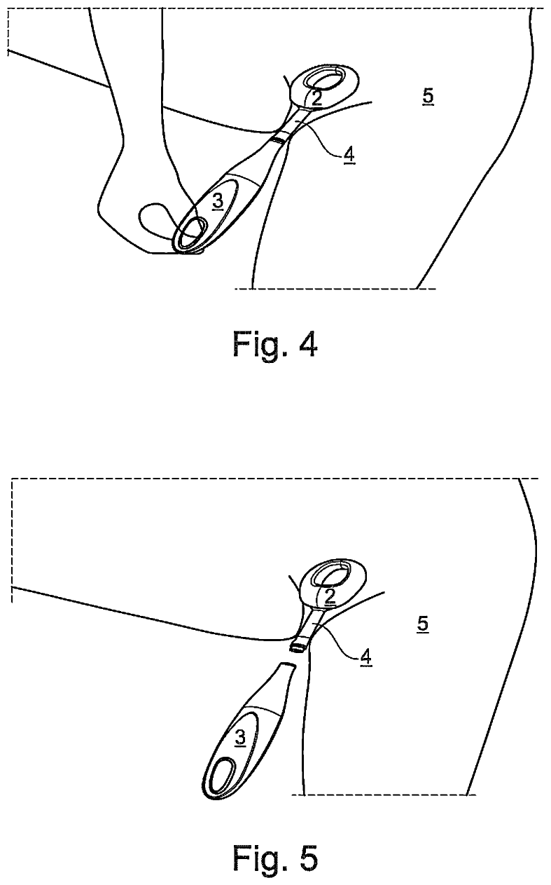

FIG. 1 shows an example in line with an embodiment according to the present invention. A system 2-4 is depicted with some of the components assembled or connected. In more detail, the system comprises a device 2 to be inserted into a user or a female user (sketched in FIGS. 4-6). The device is intended to alleviate prolapses and/or incontinence. The device can be of a very generally annular or toroidal shape with a recess that can be provided in centre section of the device. This device can be expanded in order to attach to an individual user in an optimum manner. The expansion can be realized by a fluid (e.g., gas, air, liquid, oil or any combination thereof) that is pumped into the device. The device 2 can be inflatable by a pump and can be configured to keep the inflated fluid, such as air by a valve and/or a plug and/or a cap (all not shown). Liquid can also serve as an enlarging medium. Also, a mixture of a gas with a liquid can be used in the same chambers or different chambers. The benefit would be that by inflated air the toroidal device can configure itself to the shape needed, as is also provided by the body contours of a user. One or more inner chamber(s) for air and/or liquid can be covered by a cushioning material, such as silicone or soft and/or biocompatible silicone. This could be particularly applied to parts expectedly touching sensitive regions of a user. Different pressures can alternatively or additionally be applied to the different parts of the device 2.

The device can also comprise sensors for a biofeedback of user parameters. These sensors can comprise any of one or more pressure, temperature, electromyographic (EMG) sensor(s) etc.

The device can further comprise at least one sensor for PH, motion, accelerometer, gyroscope and/or position sensing.

The device can be configured for a training of biofeedback and learning about activated muscles inside a pelvic floor.

A menstruation collecting component can also be provided to be fixedly or releasably attached to the device 2, such as a cup or other collector.

Also shown is a controlling component 3 that is configured to control any of the volume, shape, pressure, softness etc. of the device. This can be done by a pump pumping air and/or liquid from an external or internal reservoir into the device and by controlling its amount, pressure etc. A knob or any other controlling element 3a for a user can be provided in order to control those parameters. A user may activate the controlling element 3a and can add pressure and/or the amount of the fluid by the duration of activating the controlling element 3a. The controlling element 3a can also allow two ways of activation, one increasing any of the parameters mentioned (such as air pressure) or decreasing any of them. The knob 3a can be a seesaw-like switch with the two functions activated at each of two opposing ends thereof. A decreasing function can also be activated by a releasing valve, e.g., releasing pressure in the device.

A maximum parameter (such as air pressure) can be also pre-set in order to avoid and harm to a user.

The controlling component 3 can also comprise a computer or can be connected to a computer wirelessly and/or wired in order to read out sensor data, to control the device based on those sensor data and/or to inform the user about directly derived or modelled data. The latter could be an application (app) that is visualizing and/or interfacing the control by a user. Softkeys can be provided additionally or alternatively to the controlling element 3a.

The controlling component 3 can thus comprise a communication module with a wired and/or wireless transmission of information. This could be a Bluetooth sender and/or receiver. There are many alternative ways, such as other electromagnetic communication components or hardware connectors, such as USB-C, USB connectors.

The controlling component 3 can be connectable with the device by a connecting component 4. This can be a hollow tube-like connector that is transferring fluid to the device and/or away from it. Along with this a hardwire connection to sensors etc. can be also provided in the connecting component 4.

When disconnecting the connecting component 4 from the controlling component 3 (shown in other figures) the disconnection can take place between the connecting component 4 and the controlling component 3 or the device 2 or both. A check valve in the device for maintaining pressure etc. is simplifying the arrangement.

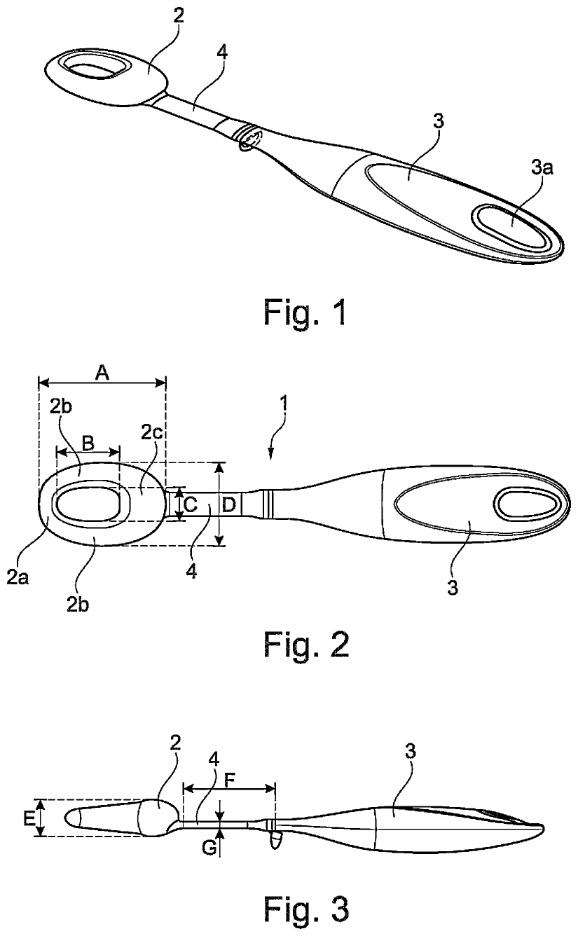

FIG. 2 shows all the above-mentioned components in from a top view. This figure shows particularly the shape of the device in an embodiment. Sides 2b of the device are symmetrical, while the tip 2a (or posterior end) is thinner than the back end 2c (or anterior end 2c). FIG. 3 shows a similar configuration in thickness (seen from the side). This enables a more comfortable and/or efficient compression or closing of a urethra of a user at least in part. A sufficient support is provided in a vagina and the part compressing tissue provides a larger surface. The relation of the cross-sections amounts to at least 1.5 (cross-section of anterior end 2c to the cross-section of the posterior end 2a), preferably at least 2 and more preferably at least 3. This is usually determined in the expanded condition of the device 2.

A recess is provided in the device accordingly so that the length B of the recess is arranged out of center to the length A of the device towards the posterior end 2a. The recess can also assist in a more elastic and comfortable seat of the device 1 by allowing an easier deformation of the ridges forming the ends as they can also deform towards or into the recess. The projection of the area of the recess from the top view shown (with the length B and the respective width) to the respective projection of the remaining part of the device (with the length A and the respective broader width) results into a ratio of at least 0.1, preferably at least 0.2 and a maximum of 0.5, preferably 0.4 and more preferably 0.3.

FIG. 3 also exemplifies the thickness E of the anterior end being considerably thicker than the one of the posterior end. Also shown are the example dimensions of the connecting component 4. The length F is intended to be long enough to implement the device properly in the vagina of the user and to still control its position and condition. It can be seen that a thickness or diameter E of the device in its expanded condition to the biggest thickness G of the connecting component 4 can be at least 2, preferably at least 3, more preferably at least 4, even more preferably at least or around 5.

FIG. 4 shows in principle the insertion and the activation of an assembly in accordance with the present invention. Particularly the dimensions and their practicability are shown. Also is apparent how a user 5 can operate the controlling component 3. The narrowing of the intermediate section of a female user with respect to the small dimensions of the diameter of the connecting assembly in contrast to the other components is also apparent.

The next FIG. 5 shows a decoupling of the controlling component 3 from the connecting component 4.

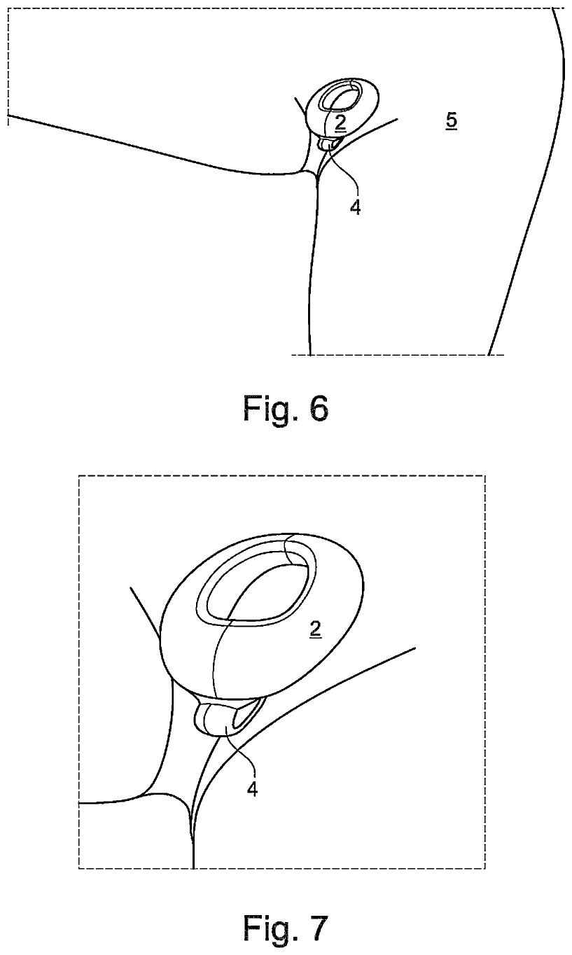

FIG. 6 and FIG. 7 in an enlarged version shown the device 2 with the connecting component 4 in folded and closed condition.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1-15. (canceled)

16. A device for alleviating prolapses and/or incontinence, comprising:
a support component, configured to provide at least partial urethral support;
wherein the support component is configured to be inserted into a vagina and to be expandable.

17. The device according to claim 16 wherein the device comprises a posterior end and an anterior end with two sides connecting the posterior end and the anterior end.

18. The device according to claim 17 wherein the anterior end is thicker and/or has a larger cross-section than the posterior end and wherein the anterior end is configured to support and/or compress a urethra from a vaginal side and the posterior end and the sides are configured to support the device in other parts of the vagina.

19. The device according to claim 16 wherein the device comprises at least one expandable volume.

20. The device according to claim 16 wherein the device comprises at least one inflatable chamber.

21. The device according to claim 16 wherein the device comprises at least one inflatable chamber within an anterior end and two sides of the device.

22. The device of claim 21 wherein the inflatable chamber is provided in each side and an anterior end of the device.

23. The device according to claim 16 wherein the device comprises three inflatable chambers, one in each one of two sides and one in an anterior end.

24. The device according to claim 16 wherein the support component comprises a body.

25. The device according to claim 16 wherein the body comprises a generally torus shape.

26. The device according to claim 16 wherein the body comprises a generally toroidal shape.

27. The device according to claim 16 wherein the body comprises an oval ring shape.

28. The device according to claim 16 wherein the body comprises an elliptical ring shape.

29. The device according to claim 16 wherein the body comprises an ovoid shape.

30. The device according to claim 16 wherein the body comprises a posterior end, an anterior end as well as two sides or side ridges) connecting the posterior end and the anterior end.

31. The device according to claim 16 further comprising a connecting component for connecting the device with a source of fluid and/or a controlling component for delivering or releasing fluid.

32. The device according to claim 16, the connecting component further comprising a releasing valve at the open end that is configured to release fluid when being activated by a user.

33. A system for alleviating prolapses and/or incontinence, comprising a device for alleviating prolapses and/or incontinence, comprising a support component, configured to provide at least partial urethral support;
wherein the support component is configured to be inserted into a vagina and to be expandable; and a controlling component that can be coupled to the device for expanding it.
